(19) United States
(12) Patent Application Publication
(10) Pub. No.: US 2022/0184235 A1
(43) Pub. Date: Jun. 16, 2022

(54) NEAR-INFRARED FLUORESCENT PROBES FOR MOLECULAR IMAGING OF BONE REPAIR

(71) Applicant: Taipeibio Co., Ltd., Taipei (TW)

(72) Inventors: Wen-Fu Lai, Taipei City (TW); Yun-Ming Wang, Kaohsiung (TW); Cheng-An Lin, Taoyuan City (TW); Li-Hsuan Chiu, Taipei City (TW); Hong-Shong Chang, Taoyuan City (TW)

(21) Appl. No.: 17/547,811

(22) Filed: Dec. 10, 2021

Related U.S. Application Data
(60) Provisional application No. 63/125,670, filed on Dec. 15, 2020.

(51) Int. Cl. A61K 49/00

(52) U.S. Cl. CPC A61K 49/0056; A61K 49/0065

(57) ABSTRACT

A probe for monitoring new bone formation and osseointegration containing an integrin α5β1 ligand conjugated to a near-infrared fluorescent dye in which the integrin α5β1 ligand is a cyclic peptide lacking an RGD sequence. Also disclosed is a bone repair and osteogenesis-monitoring probe formed of a bisphosphonate conjugated to a gold nanoparticle, the probe having a size of 1 nm to 10 nm. Further disclosed are two methods for monitoring bone formation, each of which is carried out by administering the above probes and obtaining a near-infrared fluorescence image.

### CROSS-REFERENCE TO RELATED APPLICATIONS

The present application claims the benefit of U.S. Provisional Patent Application Ser. No. 63/125,670 filed on Dec. 15, 2020, the content of which is hereby incorporated by reference in its entirety.

### BACKGROUND

Implants that promote bone repair are clinically important. The success of such implants relies on osteogenesis and osseointegration, i.e., the development of a direct bone-implant interface.

Both osteogenesis and osseointegration are typically evaluated by X-ray or by multi-detector computed tomography (“CT”). However, these imaging techniques are is associated with image artifacts that limit their utility. See Stradiotti et al. 2009, Eur. Spine J. 18 (Suppl 1):102-108. In X-ray imaging, the occurrence of artifacts depends on the scanner hardware, dense objects, catheters, specimen size, and specimen tissue density. See Fajardo et al. 2009, Bone 44:176-184; Bouxsein et al. 2010, J. Bone Min. Res. 25:1468-1486; and Lorente-Ramos et al. 2011, Am. J. Roentgenol. 196:897-904. In CT imaging, the artifacts are associated with, among other factors, image reconstruction parameters, X-ray energy, and metal composition. See Douglas-Akinwande et al. 2006, Radiographics 26 (Suppl 1):S97-110.

Such artifacts prevent clinicians from assessing whether osteogenesis is occurring following bone repair and if distortion exists between the implant and the surrounding tissue. See Stradiotti et al. and Kowada et al. 2011, J. Am. Chem. Soc. 133:17772-17776. In addition, the scan times required for CT are long and the resulting radioscintigraphic images, of a low resolution, cannot connect spatial information to cellular activity. See Zaheer et al. 2001, Nat. Biotechnol. 19:1148-1154. These artifacts and other technical issues typically lead to reduced quality of care and increased morbidity for patients suffering from bone injuries.

Clinical practice in bone repair would greatly benefit from probes and methods that can be used for precisely monitoring osteogenesis and osseointegration without the drawbacks of existing assessment tools.

### SUMMARY

To provide the benefit mentioned above, optical-specific molecular target imaging probes in the near-infra-red (“NIR”) region of the spectrum are described herein. These probes offer the advantage of high tissue penetration and low tissue auto-fluorescence, resulting in high signal-to-noise ratios during imaging.

First disclosed is a probe for monitoring new bone formation and osseointegration. The probe includes an integrin α5β1 ligand conjugated to a NIR fluorescent dye in which the integrin α5β1 ligand is a cyclic peptide lacking an RGD sequence.

Further disclosed is a probe for monitoring bone repair and osteogenesis. The probe, formed of a bisphosphonate conjugated to a gold nanoparticle, has a size of 1 nm to 10 nm.

Two methods for monitoring bone formation are also within the scope of the present invention.

A method for monitoring bone formation in a subject is provided that is carried out by administering a probe that includes a ligand for integrin α5β1 conjugated to a NIR fluorescent dye and obtaining a NIR fluorescence image at a bone repair location in the subject.

Another method for monitoring bone formation is provided. The method is performed by administering a probe that includes a gold nanoparticle conjugated to a bisphosphonate and obtaining a near-infra-red fluorescence image at a location in the subject of bone repair.

The details of several embodiments are set forth in the description and the examples below. Other features, objects, and advantages will be apparent from the detailed description, from the drawings, and also from the appended claims.

### BRIEF DESCRIPTION OF THE DRAWINGS

The description below refers to the accompanying drawings, of which:

FIG. 1A is a schematic of a conversion reaction to form CyTE777 from IR783 (Scheme 1) and a conversion reaction to form CyTE777-NHS ester from CyTE777 (Scheme 2);

FIG. 1B is a schematic of peptide synthesis, cyclization, and conjugation to form α5β1L-CyTE777;

FIG. 2A is a scanning electron microscopy image of synthesized gold nanocluster particles, i.e., nanoAu;

FIG. 2B is a plot of relative absorbance (left axis; Au@DHLA Abs) and fluorescence intensity (right axis; Au@DHLA PL) versus wavelength; and

FIG. 3 is a bar graph showing relative NIR fluorescence intensity for nanoAu (Au), nanoAu conjugated directly to pamidronate (Au+BPN), nanoAu modified with zero-length cros slinking agent 1-ethyl-3-[3-dimethyl-aminopropyl] carbodiimide is hydrochloride (Au+EDC), and nanoAu conjugated to pamidronate through EDC (Au+EDC+BPN).

### DETAILED DESCRIPTION

As summarized above, a probe for monitoring new bone formation and osseointegration is provided that includes an integrin α5β1 ligand conjugated to a NIR fluorescent dye, the ligand being a cyclic peptide lacking an RGD sequence.

The NIR fluorescent dye can be conjugated to the N-terminus of the cyclic peptide. In a specific probe, the NIR fluorescent dye is CyTE777.

The cyclic peptide can have the amino acid sequence GGCRRETAWAC (SEQ ID NO: 1) in which a bond is present between the two cystine residues.

In a preferred embodiment, the NIR fluorescent dye CyTE777 is conjugated to the N-terminal glycine residue via the amine group.

Also mentioned above is a probe for monitoring bone repair and osteogenesis. The probe, having a size of 1 nm to 10 nm, e.g., 1, 5, and 10 nm, is formed of a bisphosphonate conjugated to a gold nanoparticle.

In an alternative probe, the gold nanoparticle can be replaced with other nano-sized particles that fluoresce in the NIR region, such as semiconductor quantum dots, superparamagnetic nanoparticles (e.g., iron oxide nanoparticles), and fluorescent carbon nanoparticles (e.g., carbon dots, carbon nanotubes, graphene oxide nanoparticles, and graphene quantum dots).

The bisphosphonate is selected for its ability to bind hydroxyapatite in vivo. It can be tiludronate, clodronate, etidronate, alendronate, risedronate, pamidronate, or zolendronate. The preferred bisphosphonate is pamidronate.

In certain probes, the bisphosphonate is conjugated directly to the gold nanoparticle. In other probes, the bisphosphonate is conjugated to the gold nanoparticle via a zero-length crosslinker. An exemplary zero-length crosslinker is 1-ethyl-3-(3-dimethylaminopropyl)carbodiimide (“EDC”).

Moreover, the probe can be capped with dihydrolipoic acid.

A preferred probe is capped with dihydrolipoic acid, has a size of 1-2 nm, and is includes a gold nanoparticle conjugated via EDC to pamidronate.

The above probes can be used in either method mentioned in the SUMMARY section for monitoring bone formation.

In one method, a subject in need of bone formation monitoring is administered with a probe that includes a ligand for integrin α5β1 conjugated to a NIR fluorescent dye, and a NIR fluorescence image is obtained at a location in the subject of bone repair.

The integrin α5β1 ligand is a cyclic peptide free of an RGD sequence and can have the amino acid sequence of SEQ ID NO: 1, like the probe described above.

The NIR fluorescent dye can be CyTE777, also like the previously-described probe.

This method can also include a step of administering a probe that includes a gold nanoparticle conjugated to a bisphosphonate in which the NIR fluorescent dye and the gold nanoparticle have distinct NIR emission wavelengths. In an exemplary method, the subject is administered with both a probe capped with dihydrolipoic acid, having a size of 1-2 nm and formed of a gold nanoparticle conjugated via EDC to pamidronate, and a probe containing a cyclic peptide with the amino acid sequence of SEQ ID NO: 1 conjugated to CyTE777.

In the other method for monitoring bone formation, only the probe that includes a gold nanoparticle conjugated to a bisphosphonate is administered to the subject. Preferably, the probe is capped with dihydrolipoic acid, has a size of 1-2 nm, and is formed of a gold nanoparticle conjugated via EDC to pamidronate

Without further elaboration, it is believed that one skilled in the art can, based on the disclosure herein, utilize the present disclosure to its fullest extent. The following specific examples are, therefore, to be construed as merely descriptive, and not limitative of the remainder of the disclosure in any way whatsoever. All publications and patent documents cited herein are incorporated by reference in their entirety.

### EXAMPLES

### Example 1

### Synthesis of NIR dye CyTE-777

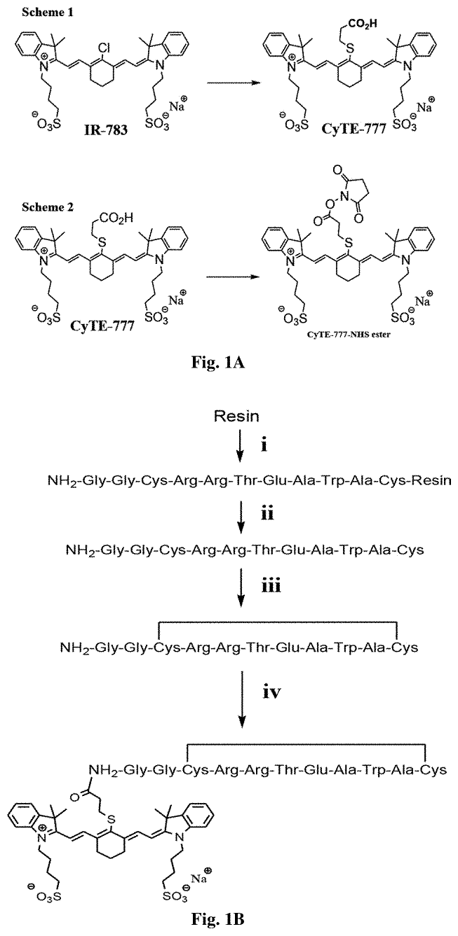

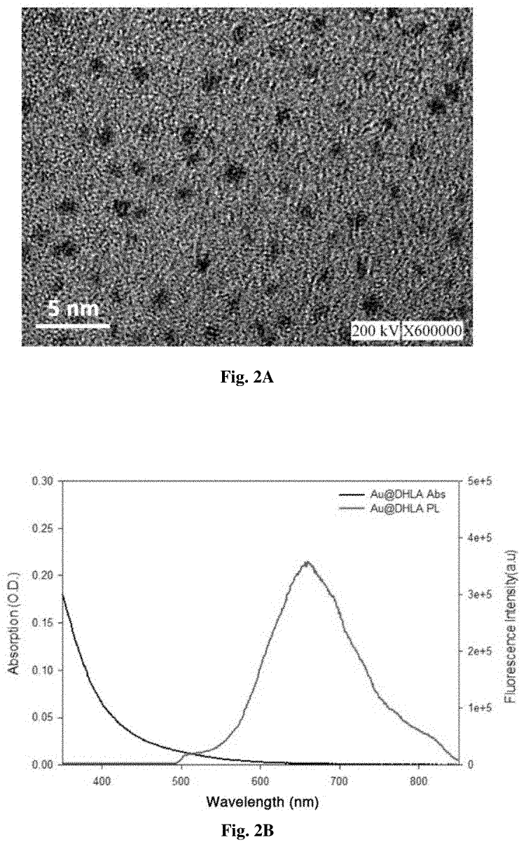

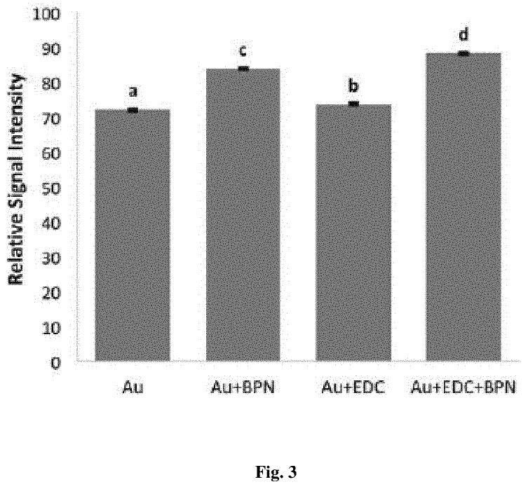

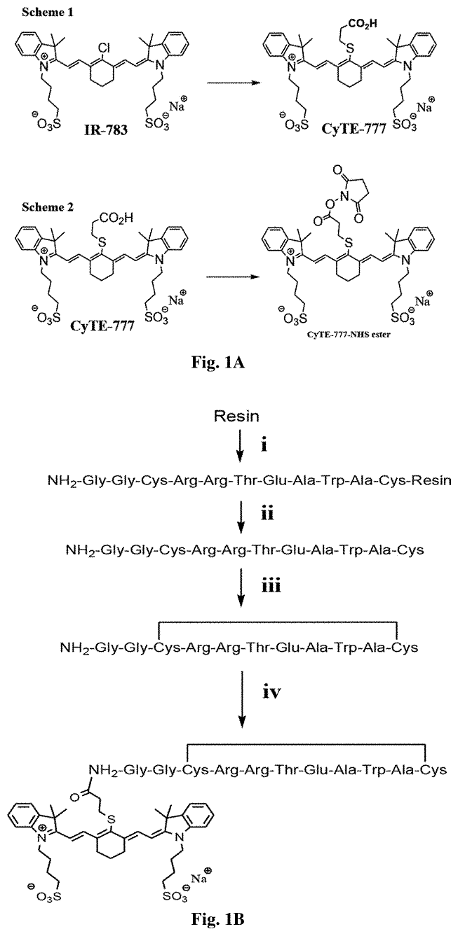

The heptamethine cyanine dye IR783, which has minimal cell toxicity, was is purchased from Sigma-Aldrich (St. Louis, MO, USA). Triethylamine (65.3 μL, 0.467 mmol) and 3-mercaptopropionic acid (40.7 μL, 0.467 mmol) were added to a solution of IR-783 (250 mg, 0.33 mmol) in 6 mL of anhydrous dimethylformamide (“DMF”). The resulting green solution was stirred in the dark at room temperature. The conversion reaction of IR783 to CyTE777 was complete after 21 h, as monitored by high-performance liquid chromatography (“HPLC”). A green solid was isolated through precipitation with ether at 4° C. and was washed with 3 mL cold ether. The precipitate was dissolved in water and dried under vacuum. The conversion of IR783 to CyTE777 is shown in FIG. 1A, scheme 1. CyTE777 had a NIR emission peak of 820 nm at an excitation of 777 nm.

¹H NMR (300 MHz, CD₃OD) results for CyTE777 were as follows: δ 8.89 (d, 2H, J=14.2 Hz), 7.49 (d, 2H, J=7.4 Hz), 7.41 (t, 2H, J=7.6 Hz), 7.34 (d, 2H, J=7.8 Hz), 7.25 (t, 2H, J=7.5 Hz), 6.32 (d, 2H, J=14.3 Hz), 4.19 (t, 4H, J=6.7 Hz), 3.06 (t, 2H, J=7.0 Hz), 2.90-2.87 (m, 4H), 2.70 (t, 4H, J=5.9 Hz), 2.56 (t, 2H, J=6.9 Hz) 2.00-1.92 (m, 10H), and 1.76 (s, 12H). HRMS-ESI [M]^{—} m/z for C₄₁H₅₁N₂O₈S₃ 795.2959 was calculated to be 795.5.

### Example 2

### Synthesis of CyTE777-N-hydroxysuccinimide ester

CyTE777 carboxylic acid (85 mg, 106.65 μmol) was introduced into a Reacti-Vial (Thermo Fisher Scientific, MA, USA) and dissolved in 500 μL of dry DMF. A solution (100 μL) of dicyclohexylcarbodiimide in dry DMF (220.05 mg, 1.0665 mmol), 186.65 μL of 4-(dimethylamino) pyridine (134.387 mg, 1.1 mmol), and N-hydroxy succinimide (“NHS,” 122.74 mg, 1.0665 mmol) were added, and the resulting reaction mixture was protected from light and stirred at room temperature for 12 h. The reaction was checked for completion by HPLC, and the resulting CyTE-777-NHS ester, i.e., CyTE777-S-ethyl-CONHS, was used without further purification. The reaction is is diagrammed in FIG. 1A, scheme 2.

### Example 3

### Synthesis of α5β1 -targeting peptide

A modified integrin α5β1 -targeting peptide, GGCRRETAWAC (SEQ ID NO: 1), was chemically synthesized by solid-phase peptide synthesis using the Fastmoc strategy is on an ABI433 A peptide synthesizer (Applied Biosystem, USA). Rink amide resin was washed with N-methylpyrrolidone (“NMP”) twice, treated twice with a solution of 20% piperidine in NMP for 1 h, and subsequently washed four times at room temperature with DMF. In a separate amino acid cartridge, Fmoc-protected amino acid (4 equiv.), O-benzotriazole-N,N,N′,N′-tetramethyl-uronium-hexafluoro-phosphate (3.6 equiv.), and N-hydroxy benzotriazole (4.0 equiv.) were dissolved in DMF (2 mL), and diisopropylethylamine (“DIEA”; 20 equiv.) was added. The active amino acid was added to the resin and allowed to react under nitrogen at room temperature for 1.5 h. The resin was drained and vortexed with acetic anhydride to promote the capping of free amines. After washing with methanol and drying under vacuum, GGCRRETAWAC was cleaved from the resin using trifluoroacetic acid/triisopropylsilane/H₂O/1,2-ethane dithiol (94.5:1:2.5:2.5, v/v/v/v) for 4 h at room temperature. The cleavage solution was precipitated in 4° C. ether, and the precipitate dissolved in ddH₂O and lyophilized.

The intramolecular disulfide bond was formed through air oxidation, followed by vigorous stirring in an aqueous ammonia solution for 3 days at room temperature. After oxidation, the aqueous solution was concentrated and filtered through a paper filter to remove the precipitate. Synthesis and disulfide bond formation of the cyclic peptide, designated as cGG*CRRETAWAC*, are shown in FIG. 1B, steps i-iii.

The purities of the synthetic peptide GGCRRETAWAC and cGG*CRRETAWAC* were confirmed by electrospray ionization mass spectrometry. The synthetic linear peptide showed a major peak of 1208.9 Da. Following cyclization via formation of an intramolecular disulfide bond between the two cystine molecules in the peptide, the major peak shifted to 1206.6 Da. The 2.8 Da decrease in molecular weight indicated that the cyclic form of the α5β1 -targeting peptide (GG*CRRETAWAC*) was dehydrogenized between the two cystine molecules.

### Example 4

### Synthesis of cGG*CRRETAWAC*-CyTE777

The conjugation of CyTE777-S-ethyl- CONHS to the NH₂ of glycine in cGG*CRRETAWAC* (designated as α5β1L) to form α5β1L-CyTE777 was carried out in a DIEA/DMF (1:9) solvent overnight at room temperature. After solvent removal, the is residue was washed with ether several times at room temperature and precipitated. The product was purified through reversed-phase HPLC. The conjugation reaction is shown in FIG. 1B, step iv.

α5β1L-CyTE777 exhibited NIR fluorescence at 820 nm at an excitation wavelength of 777 nm. The augmented fluorescence intensity indicated completed conjugation.

### Example 5

### Synthesis of a gold nanocluster-pamidronate probe

Fluorescent gold nanoclusters (“nanoAu”) were synthesized and dihydrolipoic acid (“DHLA”)-capped using a nanoparticle etching method as previously described. See Lin et al., 2009, ACS Nano 3:395-401. The particle size of the DHLA-capped nanoAu was analyzed using transmission electron microscopy (TEM) (Hitachi H-7100) operating at 80 kV using a copper grid.

Pamidronate (“PAM”) was attached to the carboxylate surface of nanoAu to form nanoAu-Pam directly or using a zero-length crosslinking agent, i.e., 1-ethyl-3-[3-dimethyl-aminopropyl] carbodiimide hydrochloride (“EDC”). Briefly, equal volumes of nanoAu (40 mM), pamidronate (3 mM), and EDC (80 mM) were mixed at room temperature for 2 h. To concentrate the nanoAu-Pam, the reaction was loaded on a 30 kDa molecular sieve (Amicon) and centrifuged at 3000 rpm for 10 min. The solution was centrifuged twice and washed with phosphate-buffered saline (“PBS”) to remove any unbound reactants. UV-visible spectroscopy was used to measure nanoAu-Pam. The purity of synthesis was checked using agarose gel electrophoresis. A UV-2510PC spectrophotometer (Labomed, Calif., USA) was used to record UV-visible absorption spectra. The fluorescence intensity of excitation spectra was recorded using an Edinburgh FL 900 CDT Time-Resolved Fluorometer (Edinburgh Analytical Instruments, Livingston, UK).

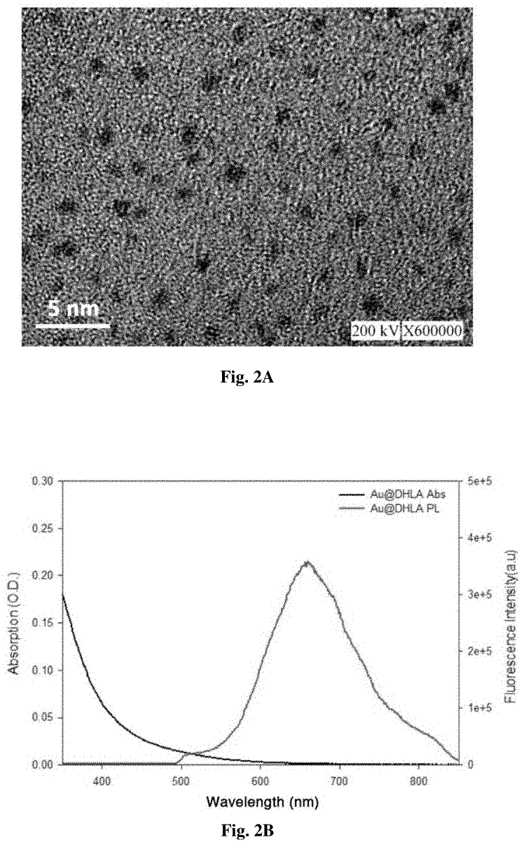

NanoAu-Pam exhibited a narrow size distribution of 1.56±0.3 nm. See FIG. 2A. This narrow size distribution resulted in a strong NIR emission at 674 nm at a broad excitation wavelength from UV to visible (350-550 nm). See FIG. 2B.

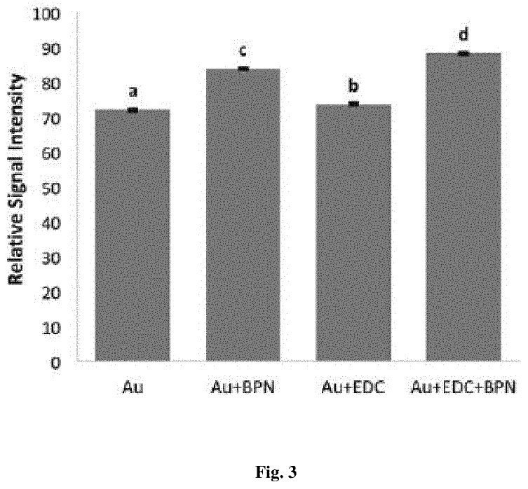

NanoAu when conjugated to Pam without or with linker EDC (nanoAu+Pam and nanoAu+EDC+Pam) showed an increase in molecular weight, as demonstrated by electrophoresis in a 2% agarose gel. Agarose gel electrophoresis also showed that the conjugates (nanoAu+Pam and nanoAu+EDC+Pam) had an increased quantum yield, as measured by fluorescence intensity. Unexpectedly, the quantum yield of the conjugate nanoAu-Pam was approximately 22.3% higher than that of nanoAu alone, with an emission peak at 674 nm. This increase in quantum yield was seen for conjugates formed with EDC or without EDC. See FIG. 3.

### Example 6

### Binding and Specificity Evaluation of nanoAu-Pam in Osteogenic Cells

To evaluate the specificity and binding cell culture, fluorescent staining of osteoblastic-differentiated mesenchymal stem cells (“MSCs”) with nanoAu-Pam was performed.

MSCs were maintained in 10 mL Dulbecco's Modified Eagles Medium with 1 g/mL glucose (“DMEM/LG”), 10% fetal bovine serum (“FBS”), and 1 x penicillin/streptomycin/fungizone. The medium was changed every four days. Cells at 80% confluence were trypsinized and passaged into new dishes at a cell density of 5×10⁵ cells/dish.

For osteogenic induction, MSCs were cultured in 24-well plates in DMEM/LG medium supplemented with 10% FBS, 100 units/mL penicillin, 100 μg/mL streptomycin, 50 μg/ml L-ascorbate-2-phosphate, 10⁻⁷ M dexamethasone and 10 mM β-glycero-phosphate at 37° C., 5% CO₂.

After 21-days of induction, the osteoblastic-differentiated MSC culture was stained with nanoAu-Pam followed by fluorescent microscopy. Briefly, 10 μM nanoAu-Pam in PBS was applied to the culture in a 24-well plate and incubated for 2 hours at 37° C., 5% CO₂. Cells were rinsed twice with PBS and fixed in 10% buffered formalin. Fixed cultures were evaluated under fluorescence microscopy.

Cells were also stained with calcein using a standard protocol to confirm calcium deposition in the culture. A 10x calcein/PBS staining solution was added to each well without medium change. The cells were incubated at 37° C., 5% CO₂ overnight, washed twice with PBS, fixed with formaldehyde, and observed by fluorescence microscopy.

The results showed that, as expected, the MSC culture showed calcium deposition is after 21 days of osteogenic induction as confirmed by positive staining with calcein. No calcein staining was seen in undifferentiated MSCs.

NanoAu-Pam also stained osteoblastic-differentiated MSCs and not undifferentiated MSCs. This result showed binding specificity of nanoAu-Pam for osteogenic cells.

### Example 7

### Binding and Specificity Evaluation of nanoAu-Pam in Prostate Cancer cell-induced hydroxyapatite deposition/calcification

To examine the specificity of nanoAu-Pam in detecting prostate cancer cell-induced hydroxyapatite (“HA”) deposition/calcification, prostate cancer cell line LNCaP was co-cultured with osteoblast cell line 7F2.

7F2 and LNCaP cells were each cultured in a-minimal essential medium supplemented with 10% FBS. Cells were cultured without any stimulatory supplements or vitamins in T25 flasks in a humidified incubator at 37° C. using a standard mixture of 95% air and 5% CO₂.

To induce HA deposition/calcification, 7F2 and LNCaP cells were co-cultured at a 1:1 ratio in 24-well plates in RPMI 1640 supplemented with 10% FBS. NanoAu-Pam was added to each well after sufficient time had elapsed to ensure HA deposition/calcification. Fluorescence of the treated cells was detected as described above.

The results showed small crystal depositions of HA, which are visible under bright-field microscopy, after 7 days of co-culture. Fluorescence of nanoAu-Pam in these cultures was seen at locations corresponding to the locations of the HA crystals.

After 28 days of culture, nanoAu-Pam showed intense fluorescence at the locations of HA deposits on the 7F2 cells that had undergone osteogenesis.

Undoubtedly, nanoAu-Pam serves as a probe to visualize osteogenic tissue using NIR spectroscopy due to the ability of pamidronate to bind to osteoblast cell surface proteins at different stages of differentiation. See Glorieux et al. 1998, N. Engl. J. Med. 339:947-952.

### Example 8

### Binding and Specificity Evaluation of α5β1L-CyTE777 in Osteogenic Cells

To determine the binding specificity of α5β1L-CyTE777, undifferentiated MSCs or osteoblastic-induced MSCs were cultured in 24-well plates in DMEM/LG supplemented with 10% FBS. α5β1L-CyTE777 was applied to the cultures to detect binding to α5β1 . Cultures incubated with α5β1L-CyTE777 were washed with PBS twice and analyzed by confocal microscopy after staining nuclei with diamidinophenylindole (“DAPI”).

The result showed that undifferentiated MSC incubated with α5β1L-CyTE777 did not show any fluorescent signal. On the other hand, osteoblastic-induced MSC incubated with α5β1L-CyTE777 showed strong NIR fluorescence at 820 nm. DAPI staining of nuclei showed that the α5β1L-CyTE777 fluorescent signal in osteoblastic-induced MSC perfectly correlated with the presence of differentiated cells.

In conclusion, NIR probes α5β1L-CyTE777 and nanoAu-Pam offer advantages including a low background noise, high signal penetration, and selective accumulation with a high fluorescence signal from osteoblasts, as compared with nontarget tissues. These novel NIR probes can detect osteogenic differentiation, osteogenesis, and bone repair with high sensitivity and specificity, making them important for the development of future diagnostic modalities that can precisely detect in vivo the extent of osseointegration of orthopedic and dental implants.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A probe for monitoring new bone formation and osseointegration, the probe comprising an integrin α5β1 ligand conjugated to a near-infra-red (NIR) fluorescent dye, wherein the integrin α5β1 ligand is a cyclic peptide free of an RGD sequence.

2. The probe of claim 1, wherein the NIR fluorescent dye is conjugated to an N-terminus of the cyclic peptide.

3. The probe of claim 2, wherein the NIR fluorescent dye is CyTE777.

4. The probe of claim 3, wherein the cyclic peptide has the sequence GGCRRETAWAC (SEQ ID NO: 1).

5. The probe of claim 1, wherein the cyclic peptide has the sequence GGCRRETAWAC (SEQ ID NO: 1).

6. The probe of claim 5, wherein the NIR fluorescent dye is CyTE777.

7. A probe for monitoring bone repair and osteogenesis, the probe comprising a bisphosphonate conjugated to a gold nanoparticle, wherein the probe has a size of 1 nm to 10 nm.

8. The probe of claim 7, wherein the bisphosphonate is conjugated directly to the gold nanoparticle.

9. The probe of claim 7, wherein the bisphosphonate is conjugated to the gold nanoparticle via a zero-length crosslinker.

10. The probe of claim 9, wherein the zero-length crosslinker is 1-ethyl-3-(3-dimethylaminopropyl)carbodiimide.

11. The probe of claim 10, wherein the probe is capped with dihydrolipoic acid.

12. The probe of claim 7, wherein the bisphosphonate is pamidronate.

13. The probe of claim 12, wherein the pamidronate is conjugated directly to the gold nanoparticle.

14. The probe of claim 12, wherein the pamidronate is conjugated to the gold nanoparticle via a zero-length crosslinker.

15. The probe of claim 14, wherein the zero-length crosslinker is 1-ethyl-3-(3-dimethylaminopropyl)carbodiimide.

16. The probe of claim 15, wherein the probe is capped with dihydrolipoic acid.

17. A method for monitoring bone formation, the method comprising identifying a subject in need of bone formation monitoring, administering a probe that includes a ligand for integrin α5β1 conjugated to a near-infra-red (NIR) fluorescent dye, and obtaining a NIR fluorescence image at a location in the subject of bone repair.

18. The method of claim 17, wherein the integrin α5β1 ligand is a cyclic peptide free of an RGD sequence.

19. The method of claim 18, wherein the cyclic peptide has the sequence GGCRRETAWAC (SEQ ID NO: 1).

20. The method of claim 19, wherein the NIR fluorescent dye is CyTE777.

21. The method of claim 17, further comprising administering a probe that includes a gold nanoparticle conjugated to a bisphosphonate, wherein the NIR fluorescent dye and the gold nanoparticle have distinct NIR emission wavelengths.

22. The method of claim 21, wherein the integrin α5β1 ligand is a cyclic peptide having the sequence GGCRRETAWAC (SEQ ID NO: 1), the NIR fluorescent dye is CyTE777, and the bisphosphonate is pamidronate.

23. A method for monitoring bone formation, the method comprising identifying a subject in need of bone formation monitoring, administering a probe that includes a gold nanoparticle conjugated to a bisphosphonate, and obtaining a near-infra-red fluorescence image at a location in the subject of bone repair.

24. The method of claim 23, wherein the probe has a size of 1 nm to 10 nm.

25. The method of claim 24, wherein the bisphosphonate is pamidronate.


SEQUENCE LISTING (1 sequence)

SEQ ID NO: 1 - protein, 11 aa, Artificial Sequence
  Synthetic peptide
GGCRRETAWAC